(19) United States
(12) Patent Application Publication
(10) Pub. No.: US 2006/0088492 A1
(43) Pub. Date: Apr. 27, 2006

(54) Hair treatment agents

(76) Inventors: Dieter Goddinger, Klein Nordende (DE); Dirk Hantrich, Hamburg (DE)

Correspondence Address: DANN DORFMAN HERRELL AND SKILLMAN;A PROFESSIONAL CORPORATION, 1601 MARKET STREET, SUITE 2400, PHILADELPHIA, PA, 19103-2307, US

(21) Appl. No.: 11/207,479

(22) Filed: Aug. 19, 2005

Related U.S. Application Data
(63) Continuation of application No. PCT/EP04/01263, filed on Feb. 11, 2004.

(30) Foreign Application Priority Data
Feb. 19, 2003 (DE) 103 07 116.4

(51) Int. Cl. A61K 8/73; A61K 8/64

(52) U.S. Cl. 424/70.13; 424/70.14

(57) ABSTRACT

The invention relates to cosmetic agents for treating, particularly for caring for, human hair. These agents contain an active combination consisting of: (A) at least one water-insoluble volatile silicone; (B) at least one cationic polymer, and; (C) at least one additional hair care substance selected from the group consisting of vitamins and/or provitamins and/or of cationically derived protein hydrolysates. The treatment of hair with a suitable formulation that contains this active substance combination leads to an improved conditioning of the hair that is made noticeable, in particular, with regard to an improved wet and dry combability and to the reduction of split ends.

### CROSS-REFERENCE TO RELATED APPLICATIONS

This application is a continuation under 35 U.S.C. §365(c) and 35 U.S.C. §120 of international application PCT/EP2004/001263, filed Feb. 11, 2004. This application also claims priority under 35 U.S.C. §119 of DE 103 07 116.4, filed Feb. 19, 2003, which is incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The invention relates to cosmetic hair treatment agents, particularly hair-care agents, based on a special combination of active substances, a process for reducing split ends as well as the use of the combination of active substances in hair treatment agents.

Nowadays, human hair of the head is treated with a variety of cosmetic preparations, which are intended to make the hair glossy and easily combable. The variety of treatments of hair, for example bleaching, dyeing, tinting and setting, can however cause unwanted damage to the hair texture. Damage to the hair texture is noticeable by a poor wet and dry combability, an increased electrostatic charge, increased brittleness, reduced maximum tear force and elongation at break of the hair, split ends and an overall worsened appearance of the hair.

A typical possibility for attenuating these grievances is offered, for example, by the aftercare of hair with compositions based on cationic polymers or water-insoluble, non-volatile silicone oils. By themselves, cationic polymers are not always able to provide a satisfactory conditioning effect, particularly concerning the suppleness and the wet and dry combability of hair.

On the other hand, insoluble, non-volatile silicone oils are widely used as good conditioners in hair-care agents, but often leave the hair with a poor, heavy and greasy touch.

(2) Description of Related Art, Including Information Disclosed Under 37 C.F.R. §§1.97 and 1.98

For these reasons, many experiments were carried out in the prior art with the aim of combining both these components in hair-care agents so as to add the positive effects and to eliminate their disadvantages. For example in EP 892 631 B1 conditioning shampoos were proposed, which comprised a combination of special cationic polymers, insoluble silicones and special surfactants, and enabled a better conditioning of the hair. Similar results are known from the documents WO 92/10162 A1, WO 94/031515 A1 and WO 95/22311 A1.

In EP 1 080 714 A1, a “2 in 1” composition was proposed, which comprised a water-soluble silicone, at least one cationic polymer and at least one surfactant, and had a better conditioning effect, particularly in regard to the suppleness and the wet and dry combability.

Nevertheless, there remains the need for hair treatment agents, which prevent or reduce hair damage resulting from external influences and aggressive cosmetic treatments such as bleaches, dyes or settings. In particular, the preparation of agents is required, which can significantly reduce or prevent split ends. At the same time, the wet and dry combability should be improved.

When using silicone oils, it is particularly important that no build-up effect, i.e. no permanent burden is placed on the hair, and that the silicone oil does not contain any non-volatile components.

Surprisingly a combination of active substances has now been found that fulfills these requirements to a large degree.

### BRIEF SUMMARY OF THE INVENTION

The subject matter of the invention therefore concerns cosmetic hair treatment agents, which comprise a combination of active substances consisting of
at least one water-insoluble volatile silicone,
at least one cationic polymer and
at least one additional hair care substance selected from the group consisting of vitamins and/or provitamins and/or of cationically derivatized protein hydrolysates.

### BRIEF DESCRIPTION OF THE SEVERAL VIEWS OF THE DRAWING(S)

Not Applicable

### DETAILED DESCRIPTION OF THE INVENTION

In the context of the invention, water-insoluble silicones are understood to mean those silicones, which have a maximum solubility of 0.5% in water at 20° C.

In the context of the invention, volatile silicones are understood to mean those, which evaporate completely and do not leave behind any residue on the substrate. Those silicones are preferred, from which 5-90% of a 10 g sample evaporates within 120 minutes on a petri dish at 60° C. Those silicones, which evaporate under these conditions to 10-50%, are particularly preferred.

Preferred inventive silicone compounds are those according to Formula (I), 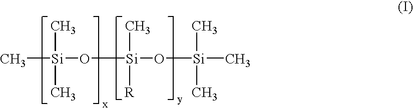
in which x stands for a number from 1 to 10, y stands for a number from 1 to 1 and R stands for an alkyl radical containing 2 to 10 C atoms.

Particularly preferred silicone compounds are those according to Formula (I), in which the numbers x and y stand for the number 1.

In the context of the invention, particularly preferred silicone components are the compounds hexyl methicone and caprylyl methicone, which, for example, are commercially available under the trade names SilCare 41M10 and SilCare 41M15 from Clariant.

The silicone components are usually used to the inventive agents in a quantity of 0.001 to 20 wt. %, preferably in a quantity of 0.1 to 10 wt. % and particularly in a quantity of 0.5 to 2 wt. %, in each case based on the total weight of the agent.

In the context of the invention, preferred cationic polymers are for example cationic cellulose derivatives, such as, e.g., a quaternized hydroxyethyl cellulose, available under the trade name Polymer JR® 400 from Amerchol, cationic starches, copolymers of diallylammonium salts and acrylamides, quaternized vinyl pyrrolidone/vinyl imidazole polymers, such as e.g., Luviquat® (BASF), condensation products of polyglycols with amines, quaternized collagen polypeptides, such as for example, lauryldimonium hydroxypropyl hydrolyzed collagen (Lamequat®L/Grünau), quaternized wheat polypeptides, polyethylene imines, cationic silicone polymers, such as amidomethicone, copolymers of adipic acid and dimethylamino hydroxypropyl diethylene triamine (Cartaretine®/Sandoz), copolymers of acrylamide and dimethyldiallylammonium chloride (Merquat® 550/Chemviron), homopolymers of dimethyidiallylammonium chloride (Merquat® 100), polyaminopolyamides, such as e.g., described in FR A 2252840 as well as their crosslinked water-soluble polymers, cationic chitin derivatives such as e.g., quaternized chitosan, optionally microcrystallinically dispersed, condensation products of dihaloalkylenes, such as e.g., dibromobutane with bis-dialkylamines, such as e.g., bis-dimethylamino-1,3-propane, cationic guar gum, such as e.g., Jaguar® CBS, Jaguar® C-17, Jaguar® C-16 from Celanese company, polymers of quaternized ammonium salts, such as e.g., Mirapol® A-15, Mirapol® AD-1, Mirapol® AZ-1 from the Miranol company.

Particularly preferred cationic polymers are cationic guar derivatives, cationic cellulose derivatives, homopolymers of dimethyldiallylammonium chloride as well as copolymers of dimethyldiallylammonium chloride and acrylamide.

The cationic polymers are usually used to the inventive agents in a quantity of 0.001 to 20 wt. %, preferably in a quantity of 0.01 to 5 wt. % and particularly in a quantity of 0.1 to 2 wt. %, in each case based on the total weight of the agent.

In a first preferred embodiment of the invention, a vitamin and/or provitamin and/or a physiologically acceptable derivative of these materials is used as the hair-care component (C).

According to the invention, such vitamins, provitamins and vitamin precursors are preferred, which are normally classified in the groups A, B, C, E, F and H.

In the group of substances designated as Vitamin A, belong retinol (vitamin A₁) as well as 3,4-didehydroretinol, (vitamin A₂). β-carotene is the provitamin of retinol. Examples of suitable vitamin A components according to the invention are vitamin A acid and its esters, vitamin A aldehyde and vitamin A alcohol as well as its esters such as the palmitate and acetate. The preparations used according to the invention preferably comprise the vitamin A components in amounts of 0.05-1 wt. % based on the total preparation.

The Vitamin B group or the vitamin B complex include inter alia
vitamin B₁ (thiamin)
vitamin B₂ (riboflavin)
vitamin B₃. The compounds nicotinic acid and nicotinamide are often included under this designation. According to the invention, nicotinamide is preferred and is comprised in the agents used according to the invention in amounts of 0.05 to 1 wt. % based on the total agent.
Vitamin B₅ (pantothenic acid, panthenol and pantolactone). In the context of this group, panthenol and/or pantolactone is preferably used. Useable derivatives of panthenol according to the invention are especially the esters and ethers of panthenol as well as cationic derivatized panthenols. Specific representatives are for example, panthenol triacetate, panthenol monoethyl ether and its monoacetate as well as the cationic panthenol derivatives disclosed in WO 92/13829. The cited compounds of the vitamin B₅ type are comprised in the agents used according to the invention in amounts of 0.05-10 wt. %, based on the total agent. Quantities of 0.1 to 5 wt. % are particularly preferred.
Vitamin B₆ (pyridoxine as well as pyridoxamine and pyridoxal).

Vitamin C (ascorbic acid). Vitamin C is preferably added to the agents used according to the invention in amounts of 0.1 to 3 wt. %, based on the total agent. Its use in the form of the palmitate ester the glucosides or phosphates can be preferred. Its use in combination with tocopherols can also be preferred.

Vitamin E (Tocopherols, especially α-tocopherol). Tocopherol and its derivatives, among which particularly the esters, such as the acetate, the nicotinate, the phosphate and the succinate, are used in the agents according to the invention preferably comprised in amounts of 0.05-1 wt. %, based on the total agent.

Vitamin F. The term “vitamin F” is usually taken to mean essential fatty acids, particularly linoleic acid, linolenic acid and arachidonoic acid.

Vitamin H. The compound (3aS,4S,6aR)-2-oxohexahydrothienol[3,4-d]-imidazole-4-valeric acid denotes Vitamin H, for which the trivial name biotin has become accepted. The agents used according to the invention preferably comprise biotin in amounts of 0.0001 to 1.0 wt. %, particularly in amounts of 0.001 to 0.01 wt. %.

The preparations used according to the invention preferably comprise vitamins, provitamins and vitamin precursors from groups A, B, E and H. Naturally, a plurality of vitamins and vitamin precursors may also be comprised at the same time.

Pantolactone, pyridoxine and its derivatives as well as nicotinamide and biotin, but particularly panthenol and its physiologically acceptable derivatives are especially preferred.

According to a second preferred embodiment of the invention, a cationically derivatized protein hydrolysate is used as the hair-care component (C).

According to the invention, animal protein hydrolysates come into consideration here, for example from collagen, milk or keratin, from plants, for example from wheat, maize, rice, potatoes, soya or almonds, from marine life, for example from fish collagen or algae, or from biotechnologically obtained protein hydrolysates. The inventive cationic derivatives based on protein hydrolysates can be obtained from the corresponding proteins by a chemical, particularly alkaline or acid hydrolysis, by an enzymatic hydrolysis and/or a combination of both types of hydrolysis. The hydrolysis of proteins generally produces a protein hydrolysate with a molecular weight distribution from about 100 daltons up to several thousand daltons. Cationic protein hydrolysates are preferred, whose base protein content has a molecular weight of 100 to 25000 daltons, preferably 250 to 5000 daltons. Moreover, cationic protein hydrolysates are understood to include quaternized amino acids and their mixtures. Quaternization of the protein hydrolysates or the amino acids is often carried out using quaternary ammonium salts such as, for example, N,N-dimethyl-N(n-alkyl)-N-(2-hydroxy-3-chloro-n-propyl)-ammonium halides. Moreover, the cationic protein hydrolysates can also be further derivatized. Typical examples of inventive cationic protein hydrolysates and derivatives are the commercially available products and those cited in the “International Cosmetic Ingredient Dictionary and Handbook”, (seventh edition 1997, The Cosmetic, Toiletry, and Fragrance Association 1101 17th Street, N.W., Suite 300, Washington, D.C. 20036-4702): cocodimonium hydroxypropyl hydrolyzed collagen, cocodimonium hydroxypropyl hydrolyzed casein, cocodimonium hydroxypropyl hydrolyzed collagen, cocodimonium hydroxypropyl hydrolyzed hair keratin, cocodimonium hydroxypropyl hydrolyzed keratin, cocodimonium hydroxypropyl hydrolyzed rice protein, cocodimonium hydroxypropyl hydrolyzed silk, cocodimonium hydroxypropyl hydrolyzed soy protein, cocodimonium hydroxypropyl hydrolyzed wheat protein, cocodimonium hydroxypropyl silk amino acids, hydroxypropyl arginine lauryl/myristyl ether hci, hydroxypropyltrimonium gelatin, hydroxypropyltrimonium hydrolyzed casein, hydroxypropyltrimonium hydrolyzed collagen, hydroxypropyltrimonium hydrolyzed conchiolin protein, hydroxypropyltrimonium hydrolyzed keratin, hydroxypropyltrimonium hydrolyzed rice bran protein, hydroxypropyltrimonium hydrolyzed silk, hydroxypropyltrimonium hydrolyzed soy protein, hydroxypropyl hydrolyzed vegetable protein, hydroxypropyltrimonium hydrolyzed wheat protein, hydroxypropyltrimonium hydrolyzed wheat protein/siloxysilicate, laurdimonium hydroxypropyl hydrolyzed soy protein, laurdimonium hydroxypropyl hydrolyzed wheat protein, laurdimonium hydroxypropyl hydrolyzed wheat protein/siloxysilicate, lauryldimonium hydroxypropyl hydrolyzed casein, lauryldimonium hydroxypropyl hydrolyzed collagen, lauryldimonium hydroxypropyl hydrolyzed keratin, lauryldimonium hydroxypropyl hydrolyzed silk, lauryldimonium hydroxypropyl hydrolyzed soy protein, steardimonium hydroxypropyl hydrolyzed casein, steardimonium hydroxypropyl hydrolyzed collagen, steardimonium hydroxypropyl hydrolyzed keratin, steardimonium hydroxypropyl hydrolyzed rice protein, steardimonium hydroxypropyl hydrolyzed silk, steardimonium hydroxypropyl hydrolyzed soy protein, steardimonium hydroxypropyl hydrolyzed vegetable protein, steardimonium hydroxypropyl hydrolyzed wheat protein, steartrimonium hydroxyethyl hydrolyzed collagen, quaternium-76 hydrolyzed collagen, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, quaternium-79 hydrolyzed wheat protein.

The cationic protein hydrolysates and derivatives of keratin, collagen, elastin, soya, milk, wheat, silk and almond are quite particularly preferred; especially preferred is hydroxypropyl hydrolyzed wheat protein, such as for example is available under the trade name Gluadin WQ from Cognis.

The agents according to the invention comprise the protein hydrolysates and their derivatives in quantities of 0.01 to 10 wt. %, based on the total agent.

Quantities of 0.1 to 5 wt. %, particularly 0.1 to 3 wt. %, are quite particularly preferred.

In a third preferred embodiment of the invention, a mixture of vitamins or provitamins and cationically derivatized protein hydrolysates is used as the component (C).

A particularly preferred mixture of components (C) according to the invention is the mixture of panthenol, or a physiologically acceptable derivative of panthenol, with hydroxypropyl hydrolyzed wheat protein.

According to the invention, the total content of component (C) in the inventive agents amounts to 0.01 to 10 wt. %, especially 0.1 to 5 wt. %, in each case based on the total weight of the agent.

According to the invention, the inventive combination of active substances can be used both in the hair treatment agents, which are rinsed out again from the hair after use, as well as in such hair treatment agents, which remain on the hair.

Exemplary preparations, which are rinsed out again from the hair after use, are shampoos, rinses and conditioners as well as setting agents and colorants. Exemplary products, which remain on the hair, are setting lotions, deep conditioners and hair-dryer waving preparations. According to the type of preparation, all suitable, customary additives and auxiliaries for the particular application can be used in addition to the inventive obligatory components.

In a preferred embodiment of the invention, the combination of active substances is made up as a shampoo.

In addition to the water excipient, the shampoos contain customary anionic, nonionic, cationic, amphoteric and/or zwitterionic surfactants, pearlizing agents, perfume components, pH regulators, dyestuffs, conservation agents, optional oils as well as viscosity regulators.

Suitable anionic surfactants for the inventive preparations are all anionic surface-active materials that are suitable for use on the human body. They are characterized by a water solubilizing anionic group, such as e.g., a carboxylate, sulfate, sulfonate or phosphate group and a lipophilic alkyl group containing about 8 to 30 C atoms. In addition, the molecule may contain glycol or polyglycol ether groups, ester, ether and amide groups as well as hydroxyl groups. Exemplary suitable anionic surfactants are, each in the form of the sodium, potassium and ammonium as well as the mono, di and trialkanolammonium salts with 2 to 4 C atoms in the alkanol group,
linear and branched fatty acids with 8 to 30 C atoms (soaps),
ether carboxylic acids of the formula R—O—(CH₂—CH₂O)ₓ—CH₂—COOH, in which R is a linear alkyl group with 8 to 30 C atoms and x=0 or 1 to 16,
acyl sarcosides with 8 to 24 C atoms in the acyl group,
acyl taurides with 8 to 24 C atoms in the acyl group,
acyl isethionates with 8 to 24 C atoms in the acyl group,
sulfosuccinic acid mono and dialkyl esters with 8 to 24 C atoms in the alkyl group and sulfosuccinic acid mono-alkylpolyoxyethyl esters with 8 to 24 C atoms in the alkyl group and 1 to 6 oxyethylene groups,
linear alkane sulfonates with 8 to 24 C atoms,
linear alpha-olefin sulfonates with 8 to 24 C atoms
alpha-sulfo fatty acid methyl esters of fatty acids with 8 to 30 C atoms,
alkyl sulfates and alkyl polyglycol ether sulfates of formula R—(OCH₂—CH₂)ₓOSO₃H, in which R is preferably a linear alkyl group with 8 to 30 C atoms and x=0 or 1 to 12,
mixtures of surface-active hydroxy sulfonates according to DE-A-37 25 030,
sulfated hydroxyalkyl polyethylenes and/or hydroxyalkylene propylene glycol ethers according to DE-A-37 23 354,
sulfonated unsaturated fatty acids with 8 to 24 C atoms and 1 to 6 double bonds according to DE-A-39 26 344,
esters of tartaric acid and citric acid with alcohols, which represent the addition products of about 2-15 molecules of ethylene oxide and/or propylene oxide on fatty alcohols with 8 to 22 C atoms,
alkyl and/or alkenyl ether phosphates of Formula (II), 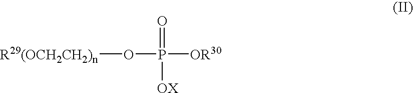
   in which R²⁹ preferably stands for an aliphatic hydrocarbon radical with 8 to 30 carbon atoms, R³⁰ stands for hydrogen, a radical (CH₂CH₂O)ₙR²⁹ or X, n for numbers between 1 and 10 and X for hydrogen, an alkali or alkaline earth metal or NR³¹R³²R³³R³⁴, with R³¹ to R³⁴, independently of each other standing for a C₁ to C₄ hydrocarbon radical,
sulfated fatty acid alkylene glycol esters of Formula (III),
   R³⁵CO(AlkO)ₙSO₃M (III)
   in which R³⁵CO stands for a linear or branched, aliphatic, saturated and/or unsaturated acyl radical with 6 to 22 C atoms, Alk for CH₂CH₂, CHCH₃CH₂ and/or CH₂CHCH₃, n for numbers from 0.5 to 5 and M for a cation, like those described in DE-OS 197 36 906.5, monoglyceride sulfates and monoglyceride ether sulfates of Formula (IV), as for example described in EP-B1-0 561 825, EP-B1-0 561 999, DE-A1 42 04 700 or by A. K. Biswas et al. in J. Am. Oil Chem. Soc. 37, 171 (1960) and F. U. Ahmed in J. Am. Oil. Chem. Soc. 67, 8 (1990) 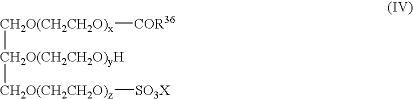
   and in which R³⁶CO stands for a linear or branched acyl radical with 6 to 22 carbon atoms, x, y and z totalize 0 or stand for numbers from 1 to 30, preferably 2 to 10, and X stands for an alkali or alkaline earth metal. In the context of the invention, typical examples of suitable monoglyceride (ether) sulfates are the reaction products of lauric acid monoglyceride, cocoa fatty acid monoglyceride, palmitic acid monoglyceride, stearic acid monoglyceride, oleic acid monoglyceride and tallow fatty acid monoglyceride as well as their ethylene oxide adducts with sulfur trioxide or chlorosulfonic acid in the form of their sodium salts. Preferably, monoglyceride sulfates of Formula (IV) are used, in which R³⁶CO stands for a linear acyl radical with 8 to 18 carbon atoms.

Preferred anionic surfactants are alkyl sulfates, alkyl polyglycol ether sulfates and ether carboxylic acids with 10 to 18 C atoms in the alkyl group and up to 12 glycol ether groups in the molecule and sulfosuccinic acid mono and dialkyl esters with 8 to 18 C atoms in the alkyl group and sulfosuccinic acid mono-alkylpolyoxyethyl esters with 8 to 18 C atoms in the alkyl group and 1 to 6 oxyethylene groups.

Zwitterionic surfactants are designated as those surface-active compounds that carry at least one quaternary ammonium group and at least one —COO⁽⁻⁾ or —SO₃⁽⁻⁾ group in the molecule. Particularly suitable zwitterionic surfactants are the so-called betaines, for example the cocoalkyldimethylammonium glycinate, N-acylaminopropyl-N,N-dimethylammonium glycinates, for example the cocoacylaminopropyldimethylammonium glycinate, and 2-alkyl-3-carboxymethyl-3-hydroxyethyl imidazolines, each with 8 to 18 carbon atoms in the alkyl or acyl groups, as well as cocoacylaminoethylhydroxyethylcarboxymethyl glycinate. A preferred zwitterionic surfactant is the fatty acid amide derivative, known under the INCI name cocoamidopropyl betaine.

The ampholytic surfactants are understood to include such surface-active compounds that apart from a C₈-C₂₄ alkyl or acyl group, contain at least one free amino group and at least one COOH or SO₃H group in the molecule, and are able to form internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycine, N-alkyltaurines, N-alkylsarcosines, 2-alkylaminopropionic acids and alkylaminoacetic acids with about 8 to 24 C atoms in each alkyl group. Particularly preferred ampholytic surfactants are N-cocoalkylamino propionate, cocoacylaminoethylamino propionate and C₁₂-C₁₈ acyl sarcosine.

Nonionic surfactants comprise, e.g., a polyol group, a polyalkylene glycol ether group or a combination of polyol and polyglycol ether groups as the hydrophilic group. Exemplary compounds are
Addition products of 2 to 50 moles ethylene oxide and/or 0 to 5 moles propylene oxide to linear and branched fatty alcohols with 8 to 30 carbon atoms, to fatty acids with 8 to 30 carbon atoms and to alkyl phenols with 8 to 15 carbon atoms in the alkyl group,
methyl or C₂-C₆ alkyl radical end blocked addition products of 2 to 50 moles ethylene oxide and/or 0 to 5 moles propylene oxide to linear and branched fatty alcohols with 8 to 30 carbon atoms, to fatty acids with 8 to 30 carbon atoms and to alkyl phenols with 8 to 15 carbon atoms in the alkyl group, such as, for example, the commercially available types Dehydol® LS, Dehydol® LT (Cognis),
C₁₂-C₃₀ fatty acid mono and diesters of addition products of 1 to 30 moles ethylene oxide on glycerin,
addition products of 5 to 60 moles ethylene oxide on castor oil and/or hydrogenated castor oil,
polyol esters of fatty acids, such as, for example, the commercial product Hydagen® HSP (Cognis) or Sovermol types (Cognis),
alkoxylated triglycerides,
alkoxylated alkyl esters of fatty acids of Formula (V),
   R³⁷CO—(OCH₂CH₂R³⁸)_{w}OR³⁹ (V)
in which R³⁷CO stands for a linear or branched, aliphatic, saturated and/or unsaturated acyl radical with 6 to 22 carbon atoms, R³⁸ for hydrogen or methyl, R³⁹ for linear or branched alkyl radicals with 1 to 4 carbon atoms and w for numbers from 1 to 20,
amine oxides,
mixed hydroxy ethers, such as described for example in DE-OS19738866,
sorbitol esters of fatty acids and addition products of ethylene oxide to sorbitol esters of fatty acids such as, e.g., the polysorbates,
sugar esters of fatty acids and addition products of ethylene oxide to sugar esters of fatty acids,
addition products of ethylene oxide to fatty acid alkanolamides and fatty amines,
fatty acid N-alkylglucamides,
alkyl polyglycosides corresponding to the general formula RO—(Z)ₓ wherein R stands for alkyl, Z for sugar and x for the number of sugar units. The alkyl polyglycosides used according to the invention may simply comprise a defined alkyl radical. However normally, these compounds are manufactured from natural fats and oils or mineral oils. In which case, the alkyl radical R are present as mixtures corresponding to the starting compounds or to each of the compounds worked up.
   Such alkyl polyglycosides are particularly preferred in which R consists
essentially of C₈-C₁₀ alkyl groups,
essentially of C₁₂-C₁₄ alkyl groups,
essentially of C₈-C₁₆ alkyl groups or
essentially of C₁₂-C₁₆ alkyl groups or
essentially of C₁₆-C₁₈ alkyl groups.

Any mono or oligosaccharide can be used as the sugar building block. Usually, sugars with 5 or 6 carbon atoms as well as the corresponding oligosaccharides are used. Such sugars are for example, glucose, fructose, galactose, arabinose, ribose, xylose, lyxose, allose, altrose, mannose, gulose, idose, talose and sucrose. Preferred sugar building blocks are glucose, fructose, galactose, arabinose und sucrose; glucose is particularly preferred.

The alkyl polyglycosides used according to the invention comprise on average 1.1 to 5 sugar units. Alkyl polyglycosides with x-values of 1.1 to 2.0 are preferred. Alkyl polyglycosides with x-values of 1.1 to 1.8 are quite particularly preferred.

The alkoxylated homologs of the cited alkyl polyglycosides can be used according to the invention. These homologs can comprise on average up to 10 ethylene oxide and/or propylene oxide units per alkyl glycoside unit.

Alkylene oxide addition products to saturated, linear fatty alcohols and fatty acids with 2 to 30 moles ethylene oxide per mole fatty alcohol or fatty acid have proved to be preferred nonionic surfactants. Preparations with excellent properties are also obtained when they comprise fatty acid esters of ethoxylated glycerin as the nonionic surfactant.

These compounds are characterized by the following parameters. The alkyl radical comprises 6 to 22 carbon atoms and may be both linear and also branched. Primary linear aliphatic radicals and aliphatic radicals, which are methyl-branched in the 2-position, are preferred. Such alkyl radicals are for example 1-octyl, 1-decyl, 1-lauryl, 1-myristyl, 1-cetyl and 1-stearyl. 1-Octyl, 1-decyl, 1-lauryl, 1-myristyl are particularly preferred. On using so-called “oxo alcohols” as starting materials, compounds with an odd number of carbon atoms in the alkyl chain preponderate.

For compounds with alkyl groups used as surfactants, they may each be pure substances. However, it is normally preferred to start with natural vegetal or animal raw materials for the manufacture of these materials, with the result that mixtures of substances are obtained, which have different alkyl chain lengths that depend on each raw material.

For surfactants, which are represented by the addition products of ethylene oxide and/or propylene oxide to fatty alcohols or derivatives of these addition products, both products with a “normal” homolog distribution as well as those with a narrow homolog distribution may be used. The term “normal” homolog distribution is understood to mean mixtures of homologs obtained from the reaction of fatty alcohols and alkylene oxide using alkali metals, alkali metal hydroxides or alkali metal alcoholates as catalysts. On the other hand, narrow homolog distributions are obtained if, e.g., hydrotalcite, alkaline earth metal salts of ether carboxylic acids, alkaline earth metal oxides, hydroxides or alcoholates are used as catalysts. The use of products with a narrow homolog distribution can be preferred.

The surfactants are used in quantities of 0.1-45 wt. %, preferably 1-30 wt. % and quite particularly preferably from 1-15 wt. %, based on the total agent.

In a preferred embodiment, nonionic, zwitterionic and/or amphoteric surfactants as well as mixtures thereof can be preferred.

According to the invention, cationic surfactants of the type quaternary ammonium compounds, the esterquats and the amido amines are likewise preferred. Preferred quaternary ammonium compounds are ammonium halides, particularly chlorides and bromides, such as alkyltrimethylammonium chlorides, dialkyldimethylammonium chlorides and trialkylmethylammonium chloride, e.g., cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, as well as the imidazolium compounds known under the INCI designations Quaternium-27 and Quaternium-83. The long alkyl chains of the abovementioned surfactants have preferably 10 to 18 carbon atoms.

Esterquats are known compounds, which both comprise at least one ester function and also a quaternary ammonium group as structural elements. Preferred esterquats are quaternized ester salts of fatty acids with triethanolamine, quaternized ester salts of fatty acids with diethanolalkylamines and quaternized ester salts of fatty acids with 1,2-dihydroxypropyidialkylamines. Such products are marketed, for example, under the trade names Stepantex®, Dehyquart® and Armocare®. The products Armocare® VGH-70, an N,N-bis(2-palmitoyloxyethyl)dimethylammonium chloride, as well as Dehyquart® F-75, Dehyquart® C-4046, Dehyquart® L80 and Dehyquart® AU 35 are examples of such esterquats.

The alkylamidoamines are normally manufactured by the amidation of natural or synthetic fatty acids and fatty acid fractions with dialkylaminoamines. According to the invention, a particularly suitable compound from this substance group is represented by stearamidopropyl dimethylamine, commercially available under the designation Tegoamid® S 18.

The agents used according to the invention preferably comprise the cationic surfactants in quantities of 0.05 to 10 wt. %, based on the total agent. Quantities of 0.1 to 5 wt. % are particularly preferred.

In a further preferred embodiment of the invention, the action of the inventive active principle can be increased by emulsifiers. Exemplary emulsifiers are
Addition products of 4 to 30 moles ethylene oxide and/or 0 to 5 moles propylene oxide to linear fatty alcohols with 8 to 22 carbon atoms, to fatty acids with 12 to 22 carbon atoms and to alkyl phenols with 8 to 15 carbon atoms in the alkyl group,
C₁₂-C₂₂ fatty acid mono and diesters of addition products of 1 to 30 moles ethylene oxide to polyols with 3 to 6 carbon atoms, particularly glycerin,
ethylene oxide and polyglycerin addition products to methylglucoside fatty acid esters, fatty acid alkanolamides and fatty acid glucamides,
C₈-C₂₂ alkyl mono and oligoglycosides and their ethoxylated analogs, wherein the degrees of oligomerization are 1.1 to 5, particularly 1.2 to 2.0 and glucose as the sugar component are preferred,
mixtures of alkyl(oligo) glucosides and fatty alcohols, for example the commercial product Montanov®68,
Addition products of 5 to 60 moles ethylene oxide on castor oil and hydrogenated castor oil,
partial esters of polyols with 3-6 carbon atoms with saturated fatty acids with 8 to 22 C atoms,
Sterols. Sterols are understood to mean a group of steroids, which carry a hydroxyl group on carbon atom 3 of the steroid skeleton and are isolated from both animal tissue (zoosterols) and vegetal fats (phytosterols). Examples of zoosterols are cholesterol and lanosterol. Examples of suitable phytosterols are ergosterol, stigmasterol and sitosterol. Sterols, the so-called mycosterols, are also isolated from fungi and yeasts.

Phospholipids. Below, principally the glucose-phospholipids are understood, which are obtained, e.g., as lecithins or phosphatidyl cholines from, e.g., egg yolk or plant seeds (e.g., soya beans).
fatty acid esters of sugars and sugar alcohols, such as sorbitol polyglycerins and polyglycerin derivatives such as, for example, polyglycerin poly 12-hydroxy stearate (commercial product Dehymuls® PGPH)
linear and branched fatty acids with 8 to 30 C atoms and their Na, K, ammonium, Ca, Mg and Zn salts.

The inventive agents preferably comprise the emulsifiers in quantities of 0.1-25 wt. %, particularly 0.5-15 wt. %, based on the total agent.

Exemplary pearlizing waxes include: alkylene glycol esters; fatty acid alkanolamides; partial glycerides; esters of polyfunctional, optionally hydroxy substituted carboxylic acids with fatty alcohols with 6 to 22 carbon atoms; fats, such as, for example fatty alcohols, fatty ketones, fatty aldehydes, fatty ethers and fatty carbonates, which have a total of at least 24 carbon atoms; ring opened products of olefin epoxides with 12 to 22 atoms with fatty alcohols with 12 to 22 carbon atoms and/or polyols with 2 to 15 carbon atoms and 2 to 10 hydroxyl groups as well as mixtures thereof.

Exemplary oils include Guerbet alcohols based on fatty alcohols with 6 to 18, preferably 8 to 10 carbon atoms, esters of linear C₆-C₂₂ fatty acids with linear alcohols, esters of branched C₆-C₁₃ carboxylic acids with linear C₆-C₂₂ fatty alcohols, esters of linear C₆-C₂₂ fatty acids with branched alcohols, especially 2-ethylhexanol, esters of linear and/or branched fatty acids with polyhydroxy alcohols (e.g., propylene glycol, dimerdiol or trimertriol) and/or Guerbet alcohols, triglycerides based on C₆-C₁₀ fatty acids, liquid mono-/di-/triglyceride mixtures based on C₆-C₁₈ fatty acids, esters of C₆-C₂₂ fatty alcohols and/or Guerbet alcohols with aromatic carboxylic acids, especially benzoic acid, vegetal oils, branched primary alcohols, substituted cyclohexanes, linear C₆-C₂₂ fatty alcohol carbonates, Guerbet carbonates, esters of benzoic acid with linear and/or branched C₆-C₂₂ alcohols (e.g., Finsolv® TN), dialkyl ethers, ring opened products of epoxidized fatty acid esters with polyols, silicone oils and/or aliphatic or naphthenic hydrocarbons.

In a further preferred embodiment of the invention, the combination of active substances is made up as a hair rinse.

In addition to the excipient water, hair rinses comprise quaternary ammonium compounds (as described in the preceding paragraphs), fatty alcohols, thickeners, emulsifiers, solubilizers, viscosity modifiers and perfume oils.

In a further preferred embodiment of the invention, the combination of active substances is made up as a setting lotion.

In addition to the excipient water, setting lotions comprise lower alcohols, surfactants, preferably nonionic surfactants (as described in the preceding paragraphs), stabilizers, cationic, nonionic and/or anionic polymers, pH modifiers as well as blowing agents, for the case of formulated foam setting lotions.

The anionic polymers, which can reinforce the action of the inventive combination of active substances, correspond to an anionic polymer, which has carboxylate and/or sulfonate groups. Exemplary anionic monomers, from which such polymers can be made, are acrylic acid, methacrylic acid, crotonic acid, maleic anhydride and 2-acrylamido-2-methylpropane sulfonic acid. Here, the acidic groups may be fully or partially present as sodium, potassium, ammonium, mono or triethanolammonium salts. Preferred monomers are 2-acrylamido-2-methylpropane sulfonic acid and acrylic acid.

Anionic polymers that comprise 2-acrylamido-2-methylpropane sulfonic acid alone or as the comonomer, have proven to be quite particularly effective; the sulfonic acid group may be fully or partially present as the sodium, potassium, ammonium, mono or triethanolammonium salt.

The homopolymer of 2-acrylamido-2-methylpropane sulfonic acid, which is commercially available, for example under the trade name Rheothik®11-80, is particularly preferred.

In this embodiment, it can be preferred to use copolymers of at least one anionic monomer and at least one nonionic monomer. Regarding the anionic monomers, reference is made to the abovementioned substances. Preferred nonionic monomers are acrylamide, methacrylamide, acrylic acid esters, methacrylic acid esters, vinyl pyrrolidone, vinyl ethers and vinyl esters.

Preferred anionic copolymers are acrylic acid-acrylamide copolymers and particularly polyacrylamide copolymers with monomers that contain sulfonic acid groups. A particularly preferred anionic copolymer consists of 70 to 55 mole % acrylamide and 30 to 45 mole % 2-acrylamido-2-methylpropane sulfonic acid, wherein the sulfonic acid group may be fully or partially present as the sodium, potassium, ammonium, mono or triethanolammonium salt. This copolymer can also be crosslinked, wherein the preferred crosslinking agents include polyolefinic unsaturated compounds such as tetraallyloxyethane, allylsucrose, allylpentaerythritol and methylene bisacrylamide. Such a polymer is comprised in the commercial product Sepigel®305 from the SEPPIC company. The use of this compound, which comprises a mixture of hydrocarbons (C₁₃-C₁₄ isoparaffin) and a nonionic emulsifier (Laureth-7) besides the polymer components, has proved to be particularly advantageous in the context of the inventive teaching.

The sodium acryloyl dimethyl taurate copolymers commercialized as a compound with isohexadecane and polysorbate 80, under the trade name Simulgel®600, have also proved to be particularly effective according to the invention.

Likewise preferred anionic homopolymers are uncrosslinked and crosslinked polyacrylic acids. Here the preferred crosslinking agents can be allyl ethers of pentaerythritol, of sucrose and of propylene. Such compounds are commercially available under the trade name Carbopol®, for example.

Copolymers of maleic anhydride and methyl vinyl ether, especially those with crosslinks are also color-conserving polymers. A maleic acid-methyl vinyl ether copolymer, crosslinked with 1,9-decadiene is commercially available under the name Stabileze® QM.

In addition, amphoteric polymers can be used as constituent polymers to increase the action of the inventive active substance. The term amphopolymers embraces not only those polymers, whose molecule includes both free amino groups and free —COOH or SO₃H groups and which are capable of forming inner salts, but also zwitterionic polymers whose molecule contains quaternary ammonium groups and —COO⁻ or —SO₃⁻ groups, and polymers containing —COOH or SO₃H groups and quaternary ammonium groups.

An example of an amphopolymer which may be used in accordance with the invention is the acrylic resin obtainable under the designation Amphomer®, which constitutes a copolymer of tert.-butylaminoethyl methacrylate, N-(1,1,3,3-tetramethylbutyl)acrylamide, and two or more monomers from the group consisting of acrylic acid, methacrylic acid and their simple esters.

Further amphoteric polymers that can be used according to the invention are the compounds cited in British Patent Application 2 104 091, European Patent Application 47 714, European Patent Application 217 274, European Patent Application 283 817 and German Offenlegungsschrift 28 17 369.

Preferred used amphoteric polymers are such polymers that are essentially composed from
a) monomers with quaternary ammonium groups of the general Formula (VI),
   R²²—CH═CR²³—CO-Z-(CₙH₂ₙ)—N⁽⁺⁾R²⁴R²⁵R²⁶A⁽⁻⁾ (VI)
   in which R²² and R²³ independently of each other stand for hydrogen or a methyl group and R²⁴, R²⁵ and R²⁶ independently of one another for alkyl groups with 1 to 4 carbon atoms, Z for a NH-group or an oxygen atom, n for a whole number from 2 to 5 and A⁽⁻⁾ is the anion of an organic or inorganic acid
and
b) monomers of carboxylic acids of the general Formula (VII),
   R²—CH═CR²⁸—COOH (VI),
   in which R²⁷ and R²⁸ independently of each other are hydrogen or methyl groups.

According to the invention, these compounds can be both used directly as well as in salt form, which is obtained by neutralization of the polymer with an alkali hydroxide, for example. With regard to the details of the preparation of these polymers, reference is specifically made to the contents of German Offenlegungsschrift 39 29 973. Quite particularly preferred are such polymers, which incorporate monomers of type (a), in which R²⁴, R²⁵ and R²⁶ are methyl groups, Z is a NH-group and A⁽⁻⁾ is a halide, methoxysulfate or ethoxysulfate ion; acrylamidopropyl trimethylammonium chloride is a particularly preferred monomer (a). Acrylic acid is preferably used as the monomer (b) in the cited polymers.

In a third variant, the inventive agents can additionally comprise nonionogenic polymers.

Suitable nonionogenic polymers are, for example:
vnyl pyrrolidone-vinyl ester copolymers, such as, for example, those marketed by BASF under the trade name Luviskol®, Luviskol® VA 64 and Luviskol® VA 73, each vinyl pyrrolidone-vinyl acetate copolymers, are likewise preferred nonionic polymers.
Cellulose ethers, such as hydroxypropyl cellulose, hydroxyethyl cellulose, and methyl hydroxypropyl cellulose, as marketed for example under the trademarks Culminal® and Benecel® (AQUALON).
Shellac
polyvinyl pyrrolidones, as are marketed, for example, under the designation Luviskol® (BASF).
siloxanes. These siloxanes can be both water-soluble and water-insoluble. Both volatile and non-volatile siloxanes are suitable, whereby non-volatile siloxanes are understood to mean such compounds with a boiling point above 200° C. at normal pressure. Preferred siloxanes are polydialkylsiloxanes, such as, for example polydimethylsiloxane, polyalkylarylsiloxanes, such as, for example polyphenylmethylsiloxane, ethoxylated polydialkylsiloxanes as well as polydialkylsiloxanes, which comprise amine and/or hydroxyl groups.
glycosidically substituted silicones according to EP 0612759 B1.

According to the invention, it is also possible for the used preparations to comprise a plurality, particularly two different polymers of the same charge and/or each with an anionic and an amphoteric and/or nonionic polymer.

The agents used according to the invention preferably comprise the polymers in quantities of 0.01 to 10 wt. %, based on the total agent. Quantities of 0.1 to 5 wt. %, particularly 0,1 to 3 wt. %, are particularly preferred.

In a further preferred embodiment of the invention, the combination of active substances is made up as a hair colorant.

In addition to the excipient water, hair colorants comprise substantive dyes and or precursors for oxidative dyes (coupling and developing components), surfactants, fatty alcohols and pH modifiers.

The developer components are normally primary aromatic amines with an additional free or substituted hydroxyl or amino group in the para or ortho position, diaminopyridine derivatives, heterocyclic hydrazones, 4-aminopyrazole derivatives as well as 2,4,5,6-tetraaminopyrimidine and derivatives thereof.

Specific representatives are, for example p-phenylenediamine, p-toluylenediamine, 2,4,5,6-tetraaminopyrimidine, p-aminophenol, N,N-bis(2′-hydroxyethyl)-p-phenylenediamine, 2(2′,5′-diaminophenyl)ethanol, 2(2′,5′-diaminophenoxy)ethanol, 4-amino-3-methylphenol, 2-aminomethyl-4-aminophenol, 4-amino-2-diethylaminomethylphenol, 2-hydroxy-4,5,6-triaminopyrimidine, 2,4-dihydroxy-5,6-diaminopyrimidine, 2,5,6-triamino-4-hydroxypyrimidine and N,N′-bis(2′-hydroxyethyl)-N,N′-bis(4′-aminophenyl)-1,3-diaminopropane-2-ol.

m-Phenylenediamine derivatives, naphthol, resorcinol and resorcinol derivatives, pyrazolones and m-aminophenol derivatives are generally used as the coupling components. Particularly suitable coupling substances are 1-naphtol, 1,5-, 2,7- and 1,7-dihydroxynaphthalene, 5-amino-2-methylphenol, m-aminophenol, resorcinol, resorcinol monomethyl ether, m-phenylenediamine, 1-phenyl-3-methylpyrazolone-5, 2,4-dichloro-3-aminophenol, 1,3-bis(2′,4′-diaminophenoxy)propane, 2-chlororesorcinol, 4-chlororesorcinol, 2-chloro-6-methyl-3-aminophenol, 2-amino-3-hydroxypyridine, 2-methylresorcinol, 5-methylresorcinol und 2-methyl-4-chloro-5-aminophenol.

Substantive dyestuffs are usually nitrophenylenediamines, nitroaminophenols, azo dyes, anthraquinones or indophenols. Preferred substantive dyestuffs are the compounds known under the international designations or trade names HC Yellow 2, HC Yellow 4, HC Yellow 5, HC Yellow 6, Basic Yellow 57, HC Orange 1, Disperse Orange 3, HC Red 1, HC Red 3, HC Red 13, HC Red BN, Basic Red 76, HC Blue 2, HC Blue 12, Disperse Blue 3, Basic Blue 7, Basic Blue 26, Basic Blue 99, HC Violet 1, Disperse Violet 1, Disperse Violet 4, Basic Violet 2, Basic Violet 14, Acid Violet 43, Disperse Black 9, Acid Black 52, Basic Brown 16 and Basic Brown 17 as well as 1,4-bis(β-hydroxyethyl)amino-2-nitrobenzene, 3-nitro-4(β-hydroxyethyl)aminophenol, 4-amino-2-nitrodiphenylamine-2′-carboxylic acid, 6-nitro-1,2,3,4-tetrahydroquinoxaline, 2-hydroxy-1,4-naphthoquinone, hydroxyethyl-2-nitrotoluidine, picramic acid, 2-amino-6-chloro-4-nitrophenol, 4-ethylamino-3-nitrobenzoic acid and 2-chloro-6-ethylamino-1-hydroxy-4-nitrobenzene. The inventive agents according to this embodiment comprise the substantive dyestuffs preferably in a quantity of 0.01 to 20 wt. %, based on the total colorant.

In addition, the inventive preparations can also comprise naturally occurring dyestuffs contained in henna red, henna neutral, henna black, camomille leaves, sandlewood, black tea, alder buckthorn bark, sage, logwood, madder root, cashoo, cedar and alkanet root.

Additional dyestuff components that are comprised in the inventive colorants can also be indoles and indolines as well as their physiologically acceptable salts. Preferred examples are 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole, 5,6-dihydroxyindole-2-carboxylic acid, 6-hydroxyindole, 6-aminoindole and 4-aminoindole. Further preferred are 5,6-dihydroxyindoline, N-methyl-5,6-dihydroxyindoline, N-ethyl-5,6-dihydroxyindoline, N-propyl-5,6-dihydroxyindoline, N-butyl-5,6-dihydroxyindoline, 5,6-dihydroxyindoline-2-carboxylic acid, 6-hydroxyindoline, 6-aminoindoline and 4-aminoindoline.

It is not required that each of the oxidation dyestuff precursors or the substantive dyestuffs be pure compounds. In fact, the inventive hair colorants, due to the manufacturing processes for the individual dyestuffs, may comprise minor quantities of even more components, in so far as they have no detrimental influence on the coloration result or that they must be excluded on other grounds, e.g., toxicological.

In regard to the useable dyestuffs in the inventive hair colorants and hair tints, reference is expressly made to the monograph of Ch. Zviak, The Science of Hair Care, chapter 7 (pages 248-250; substantive dyes) and chapter 8, pages 264-267; oxidation dyestuff precursors), published as volume 7 in the series “Dermatology” (Editor: Ch. Culnan and H. Maibach), Verlag Marcel Dekker Inc., New York, Basel, 1986, as well as the “Europäische Inventar der Kosmetik-Rohstoffe”, published by the European Union, obtainable in disk form from the Bundesverband Deutscher Industrie und Handelsunternehmen für Arzneimittel, Reformwaren und Körperpflegemittel e.V., Mannheim.

In a further preferred embodiment of the invention, the combination of active substances is made up as a waving product.

In addition to the reducing agent (thioglycolic acid, thiolactic acid), waving products generally comprise pH-modifiers, surfactants, complexants, solubilizers, fatty alcohols and perfume oils.

Exemplary additional active substances, adjuvants and additives, which can be comprised in the preferred embodiments, are
Thickeners like agar-agar, guar gum, alginates, xanthane gum, gum arabica, karaya gum, locust bean flour, linseed gums, dextrans, cellulose derivatives, e.g., methyl cellulose, hydroxyalkyl cellulose and carboxymethyl cellulose, starch fractions and derivatives of amylose, amylopectin and dextrins, clays such as e.g., bentonite or synthetic hydrocolloids such as e.g., polyvinyl alcohol,
hair conditioning compounds like phospholipids, for example soya lecithin, egg lecithin and cephalin, as well as silicone oils,
perfume oils, dimethyl isosorbitol and cyclodextrins,
solvents and solubilizers like ethanol, isopropanol, ethylene glycol, propylene glycol, glycerin and diethylene glycol,
fiber structure improvers, particularly mono, di and oligosaccharides, such as, for example glucose, galactose, fructose, fruit sugar and lactose,
conditioning active substances like paraffin oils, vegetal oils, e.g., sunflower oil, orange oil, almond oil, wheat germ oil, peach stone oil as well as quaternized amines like methyl-1-alkylamidoethyl-2-alkyl imidazolinium methosulfate,
defoamers like silicones,
dyestuffs to color the agent,
anti-dandruff active materials like piroctone olamine, zinc omadine and climbazole, active principles like allantoin and bisabolol, cholesterol,
thickeners like sugar esters, polyol esters or polyol alkyl ethers,
fats and waxes like spermaceti, beeswax, montan wax and paraffins,
fatty acid alkanolamides,
chelating agents like EDTA, NTA, β-alanine diacetic acid and phosphonic acids,
swelling and penetration agents like primary, secondary and tertiary phosphates,
opacifiers like latex, styrene/PVP copolymers and styrene/acrylamide copolymers,
pearlizing agents like ethylene glycol mono and distearate as well as PEG-3-distearate,
pigments,
blowing agents like propane-butane mixtures, N₂O, dimethyl ether, CO₂ and air,
antioxidants.

With regard to further optional ingredients and their amounts used, reference is expressly made to the relevant handbooks known to the expert, for example the monograph by K. Schrader, Grundlagen und Rezepturen der Kosmetika, 2^{nd} edition, Hüthig Buch Verlag, Heidelberg, 1989.

A second subject of the invention is a process for reducing split ends, wherein the inventive combination of active principles, comprising the components
(A) at least one water-insoluble, volatile silicone,
(B) at least one cationic polymer and
(C) at least one additional hair care substance selected from the group consisting of vitamins and/or provitamins and/or of cationically derivatized protein hydrolysates
   in a suitably formulated form is applied to the dry or damp hair and after a contact time is optionally rinsed out again.

A third subject of the invention is the use of the inventive composition of active principles, comprising
(A) at least one water-insoluble, volatile silicone,
(B) at least one cationic polymer and
(C) at least one additional hair care substance selected from the group consisting of vitamins and/or provitamins and/or of cationically derivatized protein hydrolysates,
   in hair-care agents for the reduction of split ends.

The following examples are intended to illustrate the subject of the invention more closely without limiting it in any way—all weights refer to wt. %, except when otherwise stated.

1) Shampoos

| | A | B | C |
|---|---|---|---|
| | Wt. % | Wt. % | Wt. % |
| Sodium laureth sulfate | 10 | 12 | 9 |
| Cocoamidopropyl betaine | 4 | 2 | 6 |
| Disodium cocoyl glutamate | 1 | 1 | 2 |
| Cocoamidopropyl amine oxide | 1 | | 2 |
| Coco-glucoside | | 2 | |
| Behentrimonium chloride | 1 | 1.5 | 1 |
| Cetyl alcohol | 0.3 | | 0.3 |
| Stearyl alcohol | | 0.7 | 0.3 |
| Polyquaternium-10 | 0.1 | 0.2 | 0.1 |
| Glycol distearate | | | 1 |
| Caprylyl methicone | 0.5 | 0.6 | 0.4 |
| Laurdimonium hydroxypropyl | | 0.5 | |
| hydrolyzed wheat protein | | | |
| Panthenol | 0.5 | | 0.3 |
| Conservation agent | q.s. | q.s. | q.s. |
| Perfume | q.s. | q.s. | q.s. |
| Water | Ad 100 | Ad 100 | Ad 100 |

In hair salon experiments—i.e. application of the product using the so-called half-side test with the use of 2 g product for the pre-wash and 3 g product for the main wash and subsequent technical hairdressing evaluation—the inventive shampoo showed significant advantages in the parameters wet combability, dry combability in comparison with conventional competitive products.

Moreover, the good wet combability of the inventive shampoo in comparison with conventional 2 in 1 shampoos was demonstrated by means of biophysical measurements (measurement basis: measurement of the required force for combing the strands of hair. Strands Alkinco 6634 medium blonde, measurement of 20 strands per sample and calculation of the average value).

| Product | Reduction in wet combability in % |
|---|---|
| Inventive shampoo | 56.6 |
| Competitor P (2 in 1 shampoo) | 44.5 |
| Competitor GF (2 in 1 shampoo) | 32.0 |
| Competitor WG (2 in 1 shampoo) | 42.9 |
| Competitor HE (2 in 1 shampoo) | 20.7 |

The anti-split end effect was determined using the following procedure:

Pretreatment of the Hair Strands:

The hair strands were pre-cleaned with a 10% alkaline sodium laureth sulfate solution for 15 minutes in an ultrasound bath.

Product Application:

Nine hair strands were treated per product, plus a control series. The hair strands were moistened with tap water and 1 g of product per g hair strands was worked in. After a 5-minute contact time the hair was rinsed out with cold, flowing tap water for 90 seconds. Preconditioning of the hair was carried out at 25° C. at a relative humidity of 40% for at least 12 hours.

Test Procedure:

The hair strands were combed 20000 times in an air-conditioned chamber (25° C., 40% relative humidity). After this, the damaged hair was separated from the undamaged hair by means of a fine sieve (mesh size 200 μm) and each was weighed.

Results Compared with Conventional 2 in 1 Shampoos:

| Product | Split end reduction w.r.t. control in % |
|---|---|
| Inventive shampoo | 88.0 |
| Competitor GF (2 in 1 shampoo) | 49.7 |
| Competitor LO (2 in 1 shampoo) | 55.6 |
| Competitor JLD (2 in 1 shampoo) | 21.8 |

2) Rinses

| | A | B | C |
|---|---|---|---|
| | Wt. % | Wt. % | Wt. % |
| Behentrimonium chloride | 3.75 | 3.5 | 4.2 |
| Cetyl alcohol | 2 | 3 | 1 |
| Stearyl alcohol | 1 | | 2 |
| Caprylyl methicone | 0.8 | 1.0 | 1.2 |
| Polyquaternium-10 | 0.2 | 0.1 | 0.2 |
| Panthenol | 0.5 | | |
| Laurdimonium hydroxypropyl | | 1 | 0.5 |
| hydrolyzed wheat protein | | | |
| Conservation agent | q.s. | q.s. | q.s. |
| Perfume | q.s. | q.s. | q.s. |
| Water | Ad 100 | Ad 100 | Ad 100 |

1. A composition comprising
(A) at least one water-insoluble volatile silicone,
(B) at least one cationic polymer and
(C) at least one additional hair care substance selected from the group consisting of vitamins and/or provitamins and/or of cationically derivatized protein hydrolysates.

2. The composition according to claim 1, wherein the component (A) is selected from compounds of Formula (I), 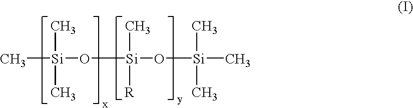
in which x stands for a number from 1 to 10, y stands for a number from 1 to 10 and R stands for an alkyl radical containing 2 to 10 C atoms.

3. The composition according to claim 1, wherein x and y are each 1.

4. The composition according to claim 1, wherein the component (A) is caprylyl methicone.

5. The composition according to claim 1, wherein the component (A) is used in a quantity of 0.001 to 20 wt. %, based on the total weight of the agent.

6. The composition according to claim 1, wherein the cationic polymer (B) is selected from the group consisting of cationic guar derivatives, cationic cellulose derivatives, homopolymers of dimethyldiallylammonium chloride or copolymers of dimethyldiallylammonium chloride and acrylamide and mixtures thereof.

7. The composition according to claim 1, wherein the cationic polymer is used in a quantity of 0.001 to 20 wt. %, based on the total weight of the agent.

8. The composition according to claim 1, wherein the component (C) is panthenol or one of its physiologically acceptable derivatives.

9. The composition according to claim 1, wherein the component (C) is selected from the group consisting of cationically derivatized keratin hydrolysates, collagen hydrolysates, elastin hydrolysates, soya protein hydrolysates, milk protein hydrolysates, wheat protein hydrolysates, silk protein hydrolysates and almond protein hydrolysates and mixtures thereof.

10. The composition according to claim 9, wherein the component (C) is hydroxypropyl hydrolyzed wheat protein.

11. The composition according to claim 1, wherein a mixture of vitamins or provitamins and cationically derivatized protein hydrolysates is used as the component (C).

12. The composition according to claim 1, wherein the content of component (C) in the total agent amounts to 0.01 to 10 wt. %.

13. A process for reducing hair damage wherein a composition comprising
(A) at least one water-insoluble volatile silicone,
(B) at least one cationic polymer and
(C) at least one additional skin and hair care substance selected from the group consisting of vitamins, provitamins, cationically derivatized protein hydrolysates, and mixtures thereof is applied to the hair and after a contact time is optionally rinsed out.

14. The process according to claim 13 wherein the type of hair damage being reduced is split ends.

15. A cosmetic agent comprising the composition as claimed in claim 1.

16. A cosmetic agent comprising the composition as claimed in claim 2.

17. A cosmetic agent comprising the composition as claimed in claim 5.

18. A cosmetic agent comprising the composition as claimed in claim 7.

19. A cosmetic agent comprising the composition as claimed in claim 12.

20. A method of hair treatment comprising the step of applying to human hair a composition comprising:
(A) at least one water-soluble volatile silicone;
(B) at least one cationic polymer; and
(C) at least one additional hair care substance selected from the group consisting of vitamins, provitamins, cationically derivatized protein hydrolysates and mixtures thereof.
